(19) United States
(12) Patent Application Publication
(10) Pub. No.: US 2025/0127650 A1
(43) Pub. Date: Apr. 24, 2025

(54) FLUID COLLECTION ASSEMBLIES INCLUDING AT LEAST ONE OF POLYURETHANE OR POLYETHYLENE FOAM

(71) Applicant: PUREWICK CORPORATION, Covington, GA (US)

(72) Inventors: Zhihui Yin, Lilburn, GA (US); Kamil Szymaniak, Dacula, GA (US); Kathleen Davis, Atlanta, GA (US); Michael Anderson, Monroe, GA (US); Brendan Tan-Fahed, Potomac, MD (US)

(21) Appl. No.: 18/835,068

(22) PCT Filed: Feb. 7, 2022
(86) PCT No.: PCT/US2022/015418

§ 371 Date: Aug. 1, 2024

(51) Int. Cl. A61F 5/455; A61F 5/453

(52) U.S. Cl. CPC A61F 5/455; A61F 5/453

(57) ABSTRACT

An example fluid collection assembly includes a fluid impermeable barrier. The fluid impermeable barrier at least defines a chamber, at least one opening. and a fluid outlet. The fluid collection assembly also includes at least one porous material disposed in the chamber. The porous material includes at least one of a polyurethane foam or a polyethylene foam. In an example, the porous material includes an outer layer disposed on an inner layer, wherein the outer layer includes the polyurethane foam and the inner layer includes the polyethylene foam.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments are directed to fluid collection assemblies including at least one of a polyurethane foam or a polyethylene foam, fluid collection systems including the same, and methods of using and forming the same. In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber. The at least one porous material includes a polyurethane foam.

In an embodiment, a fluid collection assembly is disclosed. The fluid collection assembly includes a fluid impermeable barrier at least defining a chamber, at least one opening, and a fluid outlet. The fluid collection assembly also includes at least one porous material disposed in the chamber. The at least one porous material includes a polyethylene foam.

In an embodiment, a fluid collection system is disclosed. The fluid collection system includes a fluid collection assembly. The fluid collection assembly includes a fluid impermeable barrier at least defining a chamber, at least one opening, and a fluid outlet. The fluid collection assembly also includes at least one porous material disposed in the chamber. The at least one porous material includes at least one of a polyurethane foam or a polyethylene foam. The fluid collection system also includes a fluid storage container and a vacuum source. The chamber of the fluid collection assembly, the fluid storage container, and the vacuum source are in fluid communication with each other such that a suction provided to the chamber from the vacuum source removes one or more bodily fluids from the chamber and deposits the one or more bodily fluids in the fluid storage container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIGS. 1B and 1C are cross-sectional schematics of the fluid collection assembly taken along planes 1B-1B and 1C-1C, respectively, as shown in FIG. 1A.

FIG. 2A is a cross-sectional schematic of a fluid collection assembly, according to an embodiment.

FIG. 2B is a cross-sectional schematic of the fluid collection assembly taken along plane 2B-2B shown in FIG. 2A.

FIG. 3 is a cross-sectional views of male fluid collection assembly, according an embodiment.

FIG. 4A is an isometric view of a fluid collection assembly, according to an embodiment.

FIG. 4B is a cross-sectional schematic of the fluid collection assembly taken along plane 4B-4B.

FIG. 5 is a block diagram of a fluid collection system for fluid collection, according to an embodiment.

### DETAILED DESCRIPTION

Embodiments are directed to fluid collection assemblies including at least one of a polyurethane foam or a polyethylene foam, fluid collection systems including the same, and methods of using and forming the same. An example fluid collection assembly includes a fluid impermeable barrier. The fluid impermeable barrier at least defines a chamber, at least one opening, and a fluid outlet. The fluid collection assembly also includes at least one porous material disposed in the chamber. The porous material includes at least one of a polyurethane foam or a polyethylene foam. In an example, the porous material includes an outer layer disposed on an inner layer, wherein the outer layer includes the polyurethane foam and the inner layer includes the polyethylene foam.

During use, the fluid collection assembly may be positioned on an individual such that the opening is positioned adjacent to a female urethral opening or receives a male urethral opening (i.e., penis). The individual may discharge one or more bodily fluids, such as urine, blood, or sweat. The bodily fluids may flow through the opening and into the porous material. The bodily fluids may be removed from the chamber via the fluid outlet. In an embodiment, a suction may be applied to the chamber from a vacuum source which removes the bodily fluids from the chamber.

Some conventional fluid collection assemblies include a porous material other than a polyurethane foam and/or a polyethylene foam. The porous materials of such conventional fluid collection assemblies may include a porous thin film, a gauze disposed on a polyethylene terephthalate or spun nylon fiber core, or a cover sheet disposed on a cross-lapped nonwoven filtration material. However, it has been found that the porous materials used in such conventional fluid collection assemblies can have difficultly initially receiving bodily fluids, which may cause such conventional fluid collection assemblies to leak when the individual using such conventional fluid collection assemblies discharge a large quantity of bodily fluids during a short period of time (e.g., urinates). Further, the porous materials of such conventional fluid collection assemblies are unable to quickly dry which, unless such conventional fluid collection assemblies are changed relatively frequently (e.g., changed after at least most 12 hours of use, at most 18 hours or use, or at most 24 hours of use), results in skin degradation. Other conventional fluid collection assemblies do not include a porous material in an attempt to resolve these issues associated with conventional fluid collection assemblies that include a porous material. However, such conventional fluid collection assemblies that do not include a porous material can have difficulty receiving bodily fluids and preventing pooling of the bodily fluids against the individual, either of which may cause skin degradation.

The fluid collection assemblies disclosed herein are an improvement over such conventional fluid collection assemblies at least because the fluid collection assemblies include a porous material that includes at least one of polyurethane foam or polyethylene foam. In an example, the polyurethane foam is able to quickly receive bodily fluids from the individual, even when the individual discharges a large quantity of bodily fluids over a short period of time. The polyurethane foam may also feel comfortable against the sensitive skin surrounding the urethral opening (e.g., vaginal region) of the individual. In an example, the polyethylene foam may facilitate moving the bodily fluids through the chamber of the fluid collection assembly and towards an outlet (e.g., the fluid outlet or an inlet of a conduit disposed through the fluid outlet) which allows the porous material to remain dry. When the porous material includes both the polyurethane and polyethylene foams, the porous material may quickly receive the bodily fluids, may quickly move the bodily fluids through the chamber, and may be comfortable. Further, it has been surprisingly found that the bodily fluids received into the polyurethane foam may flow easily from the polyurethane foam into the polyethylene foam and the polyethylene foam pulls bodily fluids from the polyurethane foam that would otherwise remain in the polyurethane foam. Due to the properties of the porous materials disclosed herein, the fluid collection assemblies including at least one of the polyurethane foam or the polyethylene foam may be used for prolonged periods of time without causing skin degradation. For example, the fluid collection assemblies disclosed herein including at least one of the polyurethane foam or the polyethylene foam may be used for 24 hours or more, about 30 hours or more, about 36 hours or more, about 42 hours or more, about 48 hours or more, or in ranges of about 24 hours to about 36 hours, about 30 hours to about 42 hours, or about 36 hours to about 48 hours.

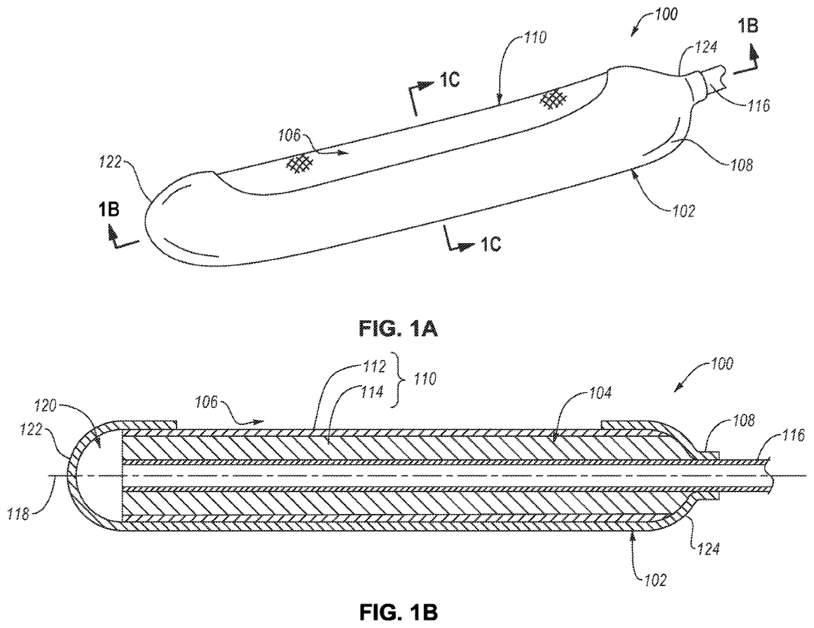

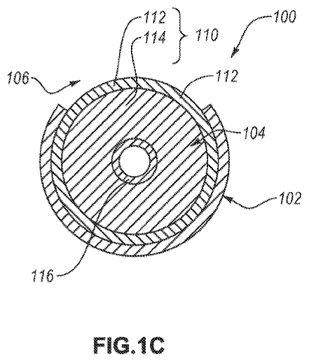

FIG. 1A is an isometric view of a fluid collection assembly 100, according to an embodiment. FIGS. 1B and 1C are cross-sectional schematics of the fluid collection assembly 100 taken along planes 1B-1B and 1C-1C, respectively, as shown in FIG. 1A. The fluid collection assembly is an example of a fluid collection assembly configured to receive bodily fluids from a female urethral opening. The fluid collection assembly 100 includes a fluid impermeable barrier 102. The fluid impermeable barrier 102 at least defines a chamber 104, at least one opening 106, and a fluid outlet 108. The fluid collection assembly 100 also includes at least one porous material 110 disposed in the chamber 104. In an embodiment, as illustrated, the porous material 110 includes an outer layer 112 extending across the opening 106 and an inner layer 114 supporting the outer layer 112 (i.e., the outer layer 112 is disposed on the inner layer 114). The outer layer 112 may include a polyurethane foam and the inner layer 114 may include a polyethylene foam though, as will be discussed in more detail below, one of the outer layer 112 or the inner layer 114 may include a material other than the polyurethane foam or the polyethylene foam, respectively.

The fluid impermeable barrier 102 at least partially defines a chamber 104 (e.g., interior region) and an opening 106. The fluid impermeable barrier 102 temporarily stores the bodily fluids in the chamber 104. The fluid impermeable barrier 102 may be formed of any suitable fluid impermeable material(s), such as a fluid impermeable polymer (e.g., silicone, polypropylene, polyethylene, polyethylene terephthalate, neoprene, a polycarbonate, etc.), a metal film, natural rubber, another suitable material, any other fluid impermeable material disclosed herein, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface of the fluid impermeable barrier 102 may be formed from a soft and/or smooth material, thereby reducing chaffing.

The opening 106 provides an ingress route for bodily fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102 thereby enabling bodily fluids to enter the chamber 104 from outside of the fluid collection assembly 100.

In some examples, the fluid impermeable barrier 102 may define a fluid outlet 108 sized to receive the conduit 116. The at least one conduit 116 may be disposed in the chamber 104 via the fluid outlet 108. The fluid outlet 108 may be sized and shaped to form an at least substantially fluid tight seal against the conduit 116 or the at least one tube thereby substantially preventing the bodily fluids from escaping the chamber 104.

As previously discussed, the fluid collection assembly 100 includes porous material 110 disposed in the chamber 104. The porous material 110 may cover at least a portion (e.g., all) of the opening 106. The porous material 110 may include an outer layer 112 and an inner layer 114. The porous material 110 is exposed to the environment outside of the chamber 104 through the opening 106.

As previously discussed, the outer layer 112 may include a polyurethane foam. In an embodiment, the polyurethane foam may by hydrophilic. The hydrophilicity of the polyurethane foam may cause the polyurethane foam to quickly pull bodily fluids therein thereby preventing or at least inhibiting leakage of bodily fluids caused by a large discharge of bodily fluids over a short period of time. The polyurethane foam may be hydrophilic when the polyurethane foam exhibits a contact angle with water (a major constituent of bodily fluids) that is about 0° to about 10°, about 5° to about 15°, about 10° to about 20°, about 15° to about 25°, about 20° to about 30°, about 25° to about 35°,about 30° to about 40°, about 35° to about 45°, about 40° to about 50°, about 45° to about 55°, about 50° to about 60°, about 55° to about 65°, about 60° to about 70°, about 65° to about 75°, about 70° to about 80°, about 75° to about 85°, or about 80° to 90°. Generally, increasing the hydrophilicity of the polyurethane foam (i.e., decreasing the contact angle between the polyurethane foam and water) increases the quantity of bodily fluids that the polyurethane foam may receive over a certain period of time. However, increasing the hydrophilicity of the polyurethane foam may increase the quantity of bodily fluids that are retained in the polyurethane foam after the polyurethane foam receives the bodily fluids. As such, the hydrophilicity of the polyurethane foam may be selected based on balancing the need to receive bodily fluids quickly while also keeping the porous material 110 dry. For example, a fluid collection assembly 100 configured to be used with an individual with a large bladder for short periods of time may include a polyurethane foam exhibiting a hydrophilicity that is greater than a polyurethane foam of a fluid collection assembly 100 configured to be used with an individual with an average to small sized bladder for long period of time.

In an embodiment, the hydrophilicity of the polyurethane foam may be an inherent property of the polyurethane foam. In an embodiment, the hydrophilicity of the polyurethane foam may be changed by at least one of impurities (e.g., fibers) or functional groups added to the polyurethane foam, otherwise treating the polyurethane foam, or coating the polyurethane foam with a material that exhibits a hydrophilicity that is different than the polyurethane foam.

The polyurethane foam defines one or more pores. The pores form passageways through the polyurethane foam through which the one or more bodily fluids may flow. In an example, the polyurethane foam exhibits a porosity of about 4 pores/cm² to about 6 pores/cm², 5 pores/cm² to about 7 pores/cm², 6 pores/cm² to about 8 pores/cm², 7 pores/cm² to about 9 pores/cm², 8 pores/cm² to about 10 pores/cm², 9 pores/cm² to about 11 pores/cm², 10 pores/cm² to about 12 pores/cm², 11 pores/cm² to about 13 pores/cm², 12 pores/cm² to about 14 pores/cm², 13 pores/cm² to about 15 pores/cm², 14 pores/cm² to about 16 pores/cm², or 15 pores/cm² to about 17 pores/cm². The porosity of the polyurethane foam may be selected based on the desired maximum quantity of bodily fluids that flow through the polyurethane foam over a certain period of time, the density of the polyurethane foam, and the surface roughness of the polyurethane foam which affects how comfortable the polyurethane foam is against the vaginal region.

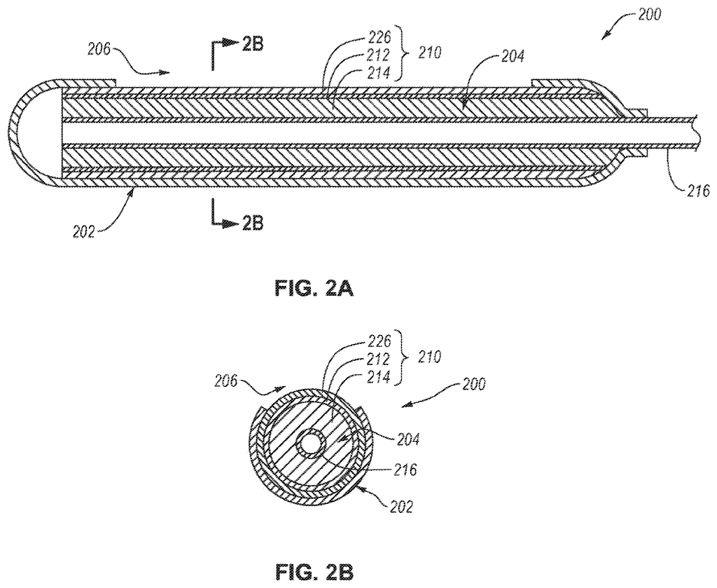

The porosity of the polyurethane foam also depends on the average lateral dimension of the pores taken in a direction that is perpendicular to a longitudinal axis 118 of the fluid collection assembly 100. The average lateral dimensions of the pores may be selected to be about 100 μm to about 300 μm, about 200 μm to about 400 μm, about 300 μm to about 500 μm, about 400 μm to about 600 μm, about 500 μm to about 700 μm, about 600 μm to about 800 μm, about 700 μm to about 900 μm, about 800 μm to about 1 mm, about 900 μm to about 1.2 mm, about 1 mm to about 1.5 mm, about 1.25 mm to about 1.75 mm, about 1.5 mm to about 2 mm, about 1.75 mm to about 2.5 mm, about 2 mm to about 3 mm, about 2.5 mm to about 3.5 mm, about 3 mm to about 4 mm, about 3.5 mm to about 4.5 mm, or about 4 mm to about 5 mm. Generally, increasing the average lateral dimension of the pores may decrease the porosity, decrease the density of the polyurethane foam, and increase the quantity of bodily fluids that may be received into the polyurethane foam over a certain period of time. However, increasing the average lateral dimension of the pores may increase the surface roughness of the polyurethane foam which makes the polyurethane foam more comfortable against the vaginal region. As such, the average lateral dimension of the pores of the polyurethane foam may be selected based on balancing these factors. For example, a fluid collection assembly that is configured to be used with an individual exhibiting a large bladder or includes a gauze layer over the polyurethane foam (as shown in FIGS. 2A and 2B) may include a polyurethane foam exhibit an average lateral dimension of the pores that is greater than the polyurethane of a fluid collection assembly that is configured to be used with an individual having an average or small bladder and/or is configured to have the polyurethane foam directly contact the vaginal region.

The polyurethane foam may exhibit an average density that is about 70 kg/m³ to about 90 kg/m³, about 80 kg/m³ to about 100 kg/m³, about 90 kg/m³ to about 110 kg/m³, about 100 kg/m³ to about 120 kg/m³, about 110 kg/m³ to about 130 kg/m³, about 120 kg/m³ to about 140 kg/m³, or about 130 kg/m³ to about 150 kg/m³. Generally, decreasing the density of the polyurethane foam may at least one of increase the porosity of the polyurethane foam, increase the average lateral dimension of the pores, increase the quantity of bodily fluids that may be received into the polyurethane foam over a certain period of time, increase the surface roughness of the polyurethane foam, and decrease the strength and toughness of the polyurethane foam. As such, the density of the polyurethane foam may be selected based on balancing these factors, as previously discussed above.

The polyurethane foam may be configured to receive bodily fluids therein at a rate of about 10 milliliters/second (“mL/s”) or greater, about 12.5 mL/s or greater, about 15 mL/s or greater, about 17.5 mL/s or greater, about 20 mL/s or greater, about 22.25 mL/s, about 25 mL/s or greater, about 27.5 mL/s or greater, about 30 mL/s or greater, about 35 mL/s or greater, about 40 mL/s or greater, or in ranges of about 10 mL/s to about 15 mL/s, about 12.5 mL/s to about 17.5 mL/s, about 15 mL/s to about 20 mL/s, about 17.5 mL/s to about 22.5 mL/s, about 20 mL/s to about 25 mL/s, about 22.5 mL/s to about 27.5 mL/s, about 25 mL/s to about 30 mL/s, about 27.5 mL/s to about 35 mL/s, or about 30 mL/s to about 40 mL/s. As previously discussed, the rate at which the polyurethane foam receives the bodily fluids depends, at least in part, on the porosity, average lateral dimension of the pores, density, and hydrophilicity of the polyurethane foam.

As previously discussed, the polyurethane foam may be formed from a hydrophilic material which may cause the polyurethane foam to retain the bodily fluids therein. To decrease the quantity of bodily fluids retained by the polyurethane foam, the outer layer 112 may be configured to be relatively thin. For example, the outer layer 112 may be configured to exhibit a thickness measured perpendicularly to the longitudinal axis 118 (e.g., measured radially) that is about 2 mm or less, about 1.5 mm or less, about 1.25 mm or less, about 1 mm or less, about 0.8 mm or less, about 0.7 mm or less, about 0.6 mm or less, about 0.5 mm or less, about 0.4 mm or less, about 0.3 mm or less, about 0.2 mm or less, or in ranges of about 0.2 mm to about 0.4 mm, about 0.3 mm to about 0.5 mm, about 0.4 mm to about 0.6 mm, about 0.5 mm to about 0.7 mm, about 0.6 mm to about 0.8 mm, about 0.7 mm to about 1 mm, about 0.8 mm to about 1.25 mm, about 1 mm to about 1.5 mm, or about 1.25 mm to about 2 mm. The relatively small thickness of the outer layer 112 decreases the overall volume of the polyurethane foam thereby decreasing the volume of bodily fluids that may be retained in the polyurethane foam. The decreasing volume of bodily fluids held within the polyurethane foam allows air flow through the chamber 104 to quickly evaporate the bodily fluids that are retained in the polyurethane foam thereby maintaining the porous material 110 dry. Further, decreasing the thickness of the outer layer 112 may allow the inner layer 114 (e.g., the polyethylene foam) to pull more bodily fluids from the polyurethane foam.

In an example, the polyurethane foam of the outer layer 112 may be formed via extrusion, such as being co-extruded with the inner layer 114. In an example, the polyurethane foam may be formed as a sheet (e.g., using a molding, casting, extrusion, or tape casting technique) and then disposed over the inner layer 114. In an example, the polyurethane foam may be molded or casted over (e.g., around) a previously formed inner layer 114.

In some embodiments, the outer layer 112 may include at least one additional material instead of or in addition to the polyurethane foam. In an example, the additional material of the outer layer 112 may include a fabric, such as a gauze (e.g., silk, linen, or cotton gauze), a woven material, a nonwoven material, another soft fabric, another smoot fabric, a polyethylene foam, or another suitable porous material. Forming the additional material of the outer layer 112 from gauze, soft fabric, and/or smooth fabric may reduce chaffing caused by the fluid collection assembly 100. In an example, the additional material of the outer layer 112 may exhibit a hydrophobicity, porosity, average lateral dimension of the pores, density, or rate at which the additional material may receive bodily fluids that is the same as or different than the hydrophobicity, porosity, average lateral dimension of the pores, density, or rate (e.g., mL/s) at which the additional material may receive bodily fluids discussed above with regards to the polyurethane foams.

As previously discussed, the porous material 110 may include an inner layer 114 disposed in the chamber 104. The inner layer 114 is configured to support the outer layer 112 since the outer layer 112 may be formed from a relatively foldable, flimsy, or otherwise easily deformable material. For example, the inner layer 114 may be positioned such that the outer layer 112 is disposed between the inner layer 114 and the fluid impermeable barrier 102. As such, the inner layer 114 may support and maintain the position of the outer layer 112.

As previously discussed, the inner layer 114 may include a polyethylene foam. In an embodiment, the polyethylene foam may by hydrophobic. The hydrophobicity of the polyethylene foam may cause the polyethylene foam to quickly push the bodily fluids or enable the bodily fluids to be drawn therethrough towards an outlet of the fluid collection assembly 100 (e.g., towards the fluid outlet 108 or the inlet of a conduit 116 disposed in the chamber 104). Pushing or drawing the bodily fluids towards the outlet of the fluid collection assembly 100 may result in the porous material 110 being dry or substantially dry shortly after the porous material 110 received the bodily fluids. The polyethylene foam may be hydrophobic when the polyethylene foam exhibits a contact angle with water (a major constituent of bodily fluids) that is about 90° to about 100°, about 95° to about 105°, about 100° to about 110°, about 105° to about 115°, about 110° to about 120°, about 115° to about 125°, about 120° to about 130°, about 125° to about 135°, about 130° to about 140°, about 135° to about 145°, about 140° to about 150°, about 145° to about 155°, about 150° to about 160°, about 155° to about 165°, about 160° to about 170°, about 165° to about 175°, or about 170° to about 180°. Generally, increasing the hydrophobicity of the polyethylene foam (i.e., increasing the contact angle between the polyethylene foam and water) allows the polyethylene foam to more quickly move the bodily fluids towards the outlet. However, increasing the hydrophobicity of the polyethylene foam may decrease the quantity of bodily fluids that may be received into the polyethylene foam during a time period. However, it has been unexpectedly found that using the polyurethane foam allows a greater quantity of bodily fluids to enter the polyethylene foam than if the porous material 110 included other materials. As such, the hydrophobicity of the polyethylene foam may be selected based on balancing the need to quickly move bodily fluids towards the outlet of the fluid collection assembly 100 while also allowing the polyethylene foam to receive the bodily fluids from the outer layer 114. For example, a fluid collection assembly 100 configured to be used with an individual with a large bladder for short periods of time may include a polyethylene foam exhibiting a hydrophobicity that is lower than a polyethylene foam of a fluid collection assembly 100 configured to be used with an individual with an average to small sized bladder for long period of time.

In an embodiment, the hydrophobicity of the polyethylene foam (i.e., the contact angle between the polyethylene foam and water) may be an inherent property of the polyethylene foam. In an embodiment, the hydrophobicity of the polyethylene foam may be changed by at least one of impurities (e.g., fibers) or functional groups added to the polyethylene foam, otherwise treating the polyethylene foam, or coating the polyethylene foam with a material that exhibits a hydrophobicity that is different than the polyethylene foam.

The polyethylene foam defines one or more pores. The pores form a passageway through the polyethylene foam through which the one or more bodily fluids may flow. In an example, the polyethylene foam exhibits a porosity of 8 pores/cm² to about 10 pores/cm², 9 pores/cm² to about 11 pores/cm², 10 pores/cm² to about 12 pores/cm², 11 pores/cm² to about 13 pores/cm², 12 pores/cm² to about 14 pores/cm², 13 pores/cm² to about 15 pores/cm², 14 pores/cm² to about 16 pores/cm², 15 pores/cm² to about 17 pores/cm², about 16 pores/cm² to about 18 pores/cm², 17 pores/cm² to about 19 pores/cm², 18 pores/cm² to about 20 pores/cm², 19 pores/cm² to about 21 pores/cm², about 20 pores/cm² to about 22 pores/cm², 21 pores/cm² to about 23 pores/cm², 22 pores/cm² to about 24 pores/cm², 23 pores/cm² to about 25 pores/cm², about 24 pores/cm² to about 26 pores/cm², 25 pores/cm² to about 27 pores/cm², 26 pores/cm² to about 28 pores/cm², 27 pores/cm² to about 29 pores/cm², or about 28 pores/cm² to about 30 pores/cm². The porosity of the polyethylene foam may be selected based on the desired quantity of bodily fluids that may be received by and flow through the polyethylene foam over a certain period of time, the average lateral dimensions of the pores, and the density of the polyethylene foam.

The porosity of the polyethylene foam also depends on the average lateral dimension of the pores taken in a direction that is perpendicular to the longitudinal axis 118 of the fluid collection assembly 100. The average lateral dimensions of the pores may be selected to be about 100 μm to about 300 μm, about 200 μm to about 400 μm, about 300 μm to about 500 μm, about 400 μm to about 600 μm, about 500 μm to about 700 μm, about 600 μm to about 800 μm, about 700 μm to about 900 μm, about 800 μm to about 1 mm, about 900 μm to about 1.2 mm, about 1 mm to about 1.5 mm, about 1.25 mm to about 1.75 mm, about 1.5 mm to about 2 mm, about 1.75 mm to about 2.5 mm, about 2 mm to about 3 mm, about 2.5 mm to about 3.5 mm, about 3 mm to about 4 mm, about 3.5 mm to about 4.5 mm, or about 4 mm to about 5 mm. Generally, increasing the average lateral dimension of the pores may decrease the porosity, decrease the density of the polyethylene foam, and increase the quantity of bodily fluids that may be received into and flow through the polyethylene foam over a certain period of time. However, increasing the average lateral dimension of the pores may weaken the polyethylene foam and allow pooling of bodily fluids therein. As such, the average lateral dimension of the pores of the polyethylene foam may be selected based on balancing these factors.

The polyethylene foam may exhibit an average density that is about 60 kg/m³ to about 80 kg/m³, about 70 kg/m³ to about 90 kg/m³, about 80 kg/m³ to about 100 kg/m³, about 90 kg/m³ to about 110 kg/m³, about 100 kg/m³ to about 120 kg/m³, about 110 kg/m³ to about 130 kg/m³, about 120 kg/m³ to about 140 kg/m³, or about 130 kg/m³ to about 150 kg/m³. Generally, decreasing the density of the polyethylene foam may at least one of increase the porosity of the polyethylene foam, increase the average lateral dimension of the pores, increase the quantity of bodily fluids that may be received into and flow through the polyethylene foam over a certain period of time, and decrease the strength and toughness of the polyethylene foam. As such, the density of the polyethylene foam may be selected based on balancing these factors, as previously discussed above.

The polyethylene foam of the inner layer 114 may be formed using any suitable technique. In an example, the polyethylene foam may be formed into a cylinder or generally cylindrical shape (e.g., generally hollow cylindrical shape) via a casting or molding technique. In an example, the polyethylene foam may be extruded, such as co-extruded with the outer layer 112. In an example, the polyethylene foam may be formed into a sheet (e.g., via casting, molding, extrusion, or tape casting) and then rolled to form a cylinder or generally cylindrical shape.

In an embodiment, the outer layer 112 includes the polyurethane foam and the inner layer 114 includes the polyethylene foam. It has been found that selecting the porous material 110 to include both the polyurethane foam and the polyethylene foam may allow the porous material 110 to receive bodily fluids quicker, move the bodily fluids towards the outlet of the fluid collection assembly 100 quicker, and better maintain the porous material 110 dry than if the porous material 110 only included one of the polyurethane foam and the polyethylene foam. For example, it has been found that bodily fluids received into the polyurethane foam are able to quickly flow from the polyurethane foam to the polyethylene foam. Further, the polyethylene foam is able to remove at least some of the bodily fluids from the polyurethane foam that would have otherwise remained in the polyurethane foam.

In an embodiment, when the porous material 110 includes the polyurethane foam and the polyethylene foam, the polyurethane foam may exhibit a porosity and density that is less than and greater than the porosity and density of the polyethylene foam, respectively. It is believed that, in such an embodiment, the bodily fluids may flow more efficiently from the polyurethane foam to the polyethylene foam.

In some embodiments, the inner layer 114 may include at least one additional material instead of or in addition to the polyethylene foam. In an example, the additional material of the inner layer 114 may include a fabric, such as a gauze (e.g., silk, linen, or cotton gauze), a woven material, a nonwoven material, a porous polymer (e.g., nylon, polyester, polyurethane, polyethylene, polypropylene, etc.) structure or an open cell foam (e.g., spun nylon fiber), any other porous material disclosed herein, or any other suitable porous material. In an example, the additional material of the inner layer 114 may exhibit a hydrophobicity, porosity, average lateral dimension of the pores, density, or rate at which the additional material may receive bodily fluids that is the same as or different than the hydrophobicity, porosity, average lateral dimension of the pores, density, or rate at which the additional material may receive bodily fluids discussed above with regards to the polyethylene foams.

In an embodiment, at least one of the outer layer 112 or the inner layer 114 may be omitted from the porous material 110.

The porous material 110 may at least substantially completely fill the portions of the chamber 104 that are not occupied by the conduit 116. In some examples, the porous material 110 may not substantially completely fill the portions of the chamber 104 that are not occupied by the conduit 116. In such an example, the fluid collection assembly 100 includes the reservoir 120 disposed in the chamber 104.

The reservoir 120 is a substantially unoccupied portion of the chamber 104. The reservoir 120 may be defined between the fluid impermeable barrier 102 and one or both of the outer layer 112 or the inner layer 114. The bodily fluids that are in the chamber 104 may flow through the porous material 110 to the reservoir 120. The reservoir 120 may retain of the bodily fluids therein.

The bodily fluids that are in the chamber 104 may flow through porous material 110 to the reservoir 120. The fluid impermeable barrier 102 may retain the bodily fluids in the reservoir 120. While depicted in the distal end region 122, the reservoir 120 may be located in any portion of the chamber 104 such as the proximal end region 124. The reservoir 120 may be located in a portion of the chamber 104 that is designed to be located in a gravimetrically low point of the fluid collection assembly when the fluid collection assembly is worn.

In some examples (not shown), the fluid collection assembly 100 may include multiple reservoirs, such as a first reservoir that is located at the portion of the chamber 104 closest to the inlet of the conduit 116 (e.g., distal end region 122) and a second reservoir that is located at the portion of the of the chamber 104 that is at or near proximal end region 124). In another example, the inner layer 114 is spaced from at least a portion of the conduit 116, and the reservoir 120 may be the space between the inner layer 114 and the conduit 116.

The conduit 116 may be at least partially disposed in the chamber 104. The conduit 116 may be used to remove the bodily fluids from the chamber 104. The conduit 116 includes at least one wall defining an inlet, an outlet (not shown) downstream from the inlet, and a passageway. The outlet of the conduit 116 may be operably coupled to a vacuum source, such as a vacuum pump for withdrawing fluid from the chamber 104 through the conduit 116. For example, the conduit 116 may extend into the fluid impermeable barrier 102 from the proximal end region 124 and may extend to the distal end region 122 to a point proximate to the reservoir 120 therein such that the inlet is in fluid communication with the reservoir 120. The conduit 116 fluidly couples the chamber 104 with the fluid storage container (not shown) or the vacuum source (not shown).

The conduit 116 may extend through a bore in the porous material 110. In an embodiment, the conduit 116 extends from the fluid outlet 108, through the bore, to a location that is proximate to the reservoir 120. In such an embodiment, the inlet may not extend into the reservoir 120 and, instead, the inlet may be disposed within the porous material 110 or at a terminal end thereof. For example, an end of the conduit 116 may be coextensive with or recessed within the porous material 110. In an embodiment, the conduit 116 is at least partially disposed in the reservoir 120 and the inlet may be extended into or be positioned in the reservoir 120. The bodily fluids collected in the fluid collection assembly 100 may be removed from the chamber 104 via the conduit 116.

Locating the inlet of the conduit 116 at or near a location expected to be the gravimetrically low point of the chamber 104 when worn by an individual enables the conduit 116 to receive more of the bodily fluids than if inlet was located elsewhere and reduce the likelihood of pooling (e.g., pooling of the bodily fluids may cause microbe growth and foul odors). For instance, the bodily fluids in the porous material 110 may flow in any direction due to capillary forces. However, the bodily fluids may exhibit a preference to flow in the direction of gravity, especially when at least a portion of the porous material 110 is saturated with the bodily fluids. Accordingly, one or more of the inlet of the conduit 116 or the reservoir 120 may be located in the fluid collection assembly 100 in a position expected to be the gravimetrically low point in the fluid collection assembly 100 when worn by an individual, such as the distal end region 122.

The conduit 116 is configured to allow the vacuum source (not shown) to be in fluid communication with the chamber 104 (e.g., the reservoir 120). As the vacuum source applies a vacuum/suction in the conduit 116, the bodily fluids in the chamber 104 (e.g., at the distal end region 122 such as in the reservoir 120) may be drawn into the inlet of the conduit 116 and out of the fluid collection assembly 100 via the conduit 116. In some examples, the conduit 116 may be frosted or opaque (e.g., black) to obscure visibility of the bodily fluids therein.

The fluid collection assemblies disclosed herein may include one or more additional layers instead of or in addition to at least one of the outer layer or the inner layer. FIG. 2A is a cross-sectional schematic of a fluid collection assembly 200, according to an embodiment. FIG. 2B is a cross-sectional schematic of the fluid collection assembly 200 taken along plane 2B-2B shown in FIG. 2A. Except as otherwise disclosed herein, the fluid collection assembly 200 is the same or substantially similar to any of the fluid collection assemblies disclosed herein. For example, the fluid collection assembly 200 may include a fluid impermeable barrier 202 at least defining a chamber 204 and at least one opening 206. The fluid collection assembly 200 may also include at least one porous material 210 disposed in the chamber 204. The porous material 210 may include an outer layer 212 and an inner layer 214.

The porous material 210 may include one or more additional layers 226 instead of or in addition to the outer layer 212 or the inner layer 214. In an embodiment, as illustrated, the additional layer 226 may be disposed on the outer layer 212 and extend across the opening 206. In such an embodiment, the additional layer may include a gauze or other material that may be comfortable against the vaginal region of the individual or more visually pleasing than the outer layer 212. It is noted that the thickness of the additional layer 226 may be minimized (e.g., about 1 mm or less or about 0.5 mm or less) such that any affect the additional layer 226 has on the porous material 210 receiving the bodily fluids discharged by the individual are minimized. In an embodiment, the additional layer 226 is positioned between the outer layer 212 and the inner layer 214. In an embodiment, the additional layer 226 may be positioned between the inner layer 214 and the conduit 216.

In some embodiments, the porous material 210 may include one or more additional layers. For example, the porous material 210 may include one or more additional layer disposed at least one of on the outer layer 212, between the outer layer 212 and the inner layer 214, or within the inner layer 214. The one or more additional layers may include any of the porous materials disclosed herein.

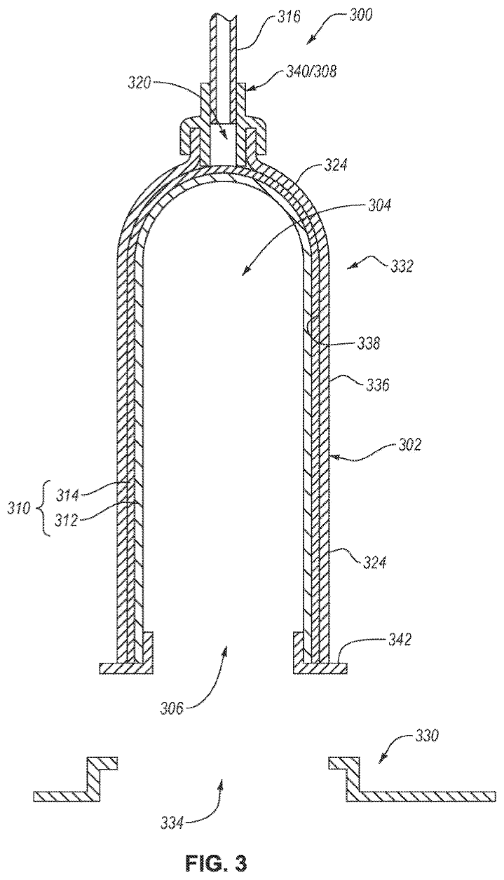

The fluid collection assemblies shown in FIGS. 1A-2B are examples of female fluid collection assemblies that are configured to collect bodily fluids from females (e.g., collect urine from a female urethra). However, the fluid collection assemblies, systems, and methods disclosed herein may include male fluid collection assemblies shaped, sized, and otherwise configured to collect bodily fluids from males (e.g., collect urine from a male urethra). FIG. 3 is a cross-sectional views of male fluid collection assembly 300, according an embodiment.

Referring to FIG. 3, the fluid collection assembly 300 includes a base 330 (e.g., annular base) and a sheath 332. The base 330 is sized, shaped, and made of a material to be coupled to skin that surrounds the male urethral opening (e.g., penis) and have the male urethral opening positioned therethrough. For example, the base 330 may define an aperture 334. The base 330 is sized and shaped to be positioned around the male urethral opening (e.g., positioned around and/or over the penis) and the aperture 334 may be configured to have the male urethral opening positioned therethrough. The base 330 may also be sized, shaped, made of a material, or otherwise configured to be coupled (e.g., adhesively attached, such as with a hydrogel adhesive) to the skin around the male urethral opening (e.g., around the penis). In an example, the base 330 may exhibit the general shape or contours of the skin surface that the base 330 is selected to be coupled with. The base 330 may be flexible thereby allowing the base 330 to conform to any shape of the skin surface. The base 330 also defines a hollowed region that is configured to receive (e.g., seal against) the sheath 332. The base 330 is located at a proximal end region 324 (with respect to a wearer) of the fluid collection assembly 300.

The sheath 332 includes (e.g., may be formed from) a fluid impermeable barrier 302 that is sized and shaped to fit into the hollowed region of the base 330. For example, the sheath 332 may be generally tubular or cup-shaped, as shown. The generally tubular or cup-shaped fluid impermeable barrier 302 may at least partially define the outer surface 336 of the sheath 332. The fluid impermeable barrier 302 may be similar or identical to and of the fluid impermeable barriers disclosed herein, in one or more aspects. For example, the fluid impermeable barrier 302 may be constructed of any of the materials disclosed herein for the fluid impermeable barrier. The fluid impermeable barrier 302 at least partially defines the chamber 304. For example, the inner surface 338 of the fluid impermeable barrier 302 at least partially defines the perimeter of the chamber 304. The chamber 304 may at least temporarily retain bodily fluids therein. As shown, the fluid collection assembly 300 may include the porous material 310 therein. The porous material 310 may be similar or identical any of the porous materials disclosed herein, in one or more aspects. For example, the porous material 310 may include one or more of an inner layer 312, an outer layer 314, or one or more additional layers. The inner layer 312 may include polyurethane and/or the outer layer 314 may include polyethylene. The fluid impermeable barrier 302 may also define an opening 306 extending through the fluid impermeable barrier 302 that is configured to have a male urethral opening positioned therethrough.

The sheath 332 also includes at least a portion of the conduit 316 therein, such as at least partially disposed in the chamber 304. For example, the conduit 316 may extend from the sheath 332 at the distal end region 322 to a proximal end region 324 at least proximate to the opening 306. The proximal end region 324 may be disposed near or on the skin around the male urethral opening (e.g., on the penis or pubic area therearound). Accordingly, when an individual lays on their back, bodily fluids (e.g., urine) may aggregate near the opening 306 against the skin of the subject. The bodily fluids may be removed from the chamber 304 via the conduit 316.

In some examples, the fluid impermeable barrier 302 may be constructed of a material and/or have a thickness that allows the sheath 332 to collapse when placed under vacuum, such as to remove air around a penis in the fluid collection assembly 300 during use. In such examples, the conduit 316 may extend only to or into the distal end region 322 in the chamber 304 (e.g., not through to the area adjacent the opening 306). In such examples, urine may be collected and removed from the fluid collection assembly 300

In an example, portions of the chamber 304 may be substantially empty due to the varying sizes and rigidity of the male penis. However, in some examples, the outermost regions of the chamber 304 (e.g., periphery of the interior regions of the sheath 332) may include the porous material 310. For example, the porous material 310 may be bonded to the inner surface 338 of the fluid impermeable barrier 302. The porous material 310 may be positioned (e.g., at the distal end of the chamber 304) to blunt a stream of urine from the male urethral opening thereby limiting splashing and/or to direct the bodily fluids to a selected region of the chamber 304. Since the chamber 304 is substantially empty (e.g., substantially all of the chamber 304 forms a reservoir), the bodily fluids are likely to pool at a gravimetrically low point of the chamber 304. The gravimetrically low point of the chamber 304 may be at an intersection of the skin of an individual and the fluid collection assembly 300, a corner formed in the sheath 332, or another suitable location depending on the orientation of the wearer.

As previously discussed, the porous material 310 may include one or more of the outer layer 312 or the inner layer 314. One or more of outer layer 312 or the inner layer 314 may be disposed between the fluid impermeable barrier 302 and a penis inserted into the chamber 304. The outer layer 312 may be positioned between the fluid impermeable barrier 302 and a penis inserted into the chamber 304, such as between the inner layer 314 and penis of a wearer as shown. The inner layer 314 may be positioned between the outer layer 312 and the fluid impermeable barrier 302. The inner surface 338, optionally including the end of the chamber 304 substantially opposite the opening 306, may be covered with one or both outer layer 312 or the outer layer 314. The outer layer 312 or the inner layer 314 may be affixed (e.g., adhered) to the fluid impermeable barrier 302. The outer layer 312 and the inner layer 314 may be affixed to each other. In some examples, the porous material 310 only includes the outer layer 312 or the inner layer 314.

The fluid collection assembly 300 includes a cap 340 at a distal end region 322. The cap 340 defines an interior channel through which the bodily fluids may be removed from the fluid collection assembly 300. The interior channel is in fluid communication with the chamber 304. The cap 340 may be disposed over at least a portion of the distal end region 322 of one or more of the fluid impermeable barrier 302 or the porous material 310. The cap 340 may be made of a polymer, rubber, or any other fluid impermeable material. The cap 340 may be attached to one or more of the fluid impermeable barrier 302, the porous material 310, or the conduit 316. The cap 340 may cover at least a portion of the distal end region 322 of the fluid collection assembly 300. The cap 340 may define a fluid outlet 308 that is sized and configured to receive and fluidly seal against the conduit 316. The conduit 316 may extend a distance within or through the cap 340, such as to the porous material 310, through the porous material 310, or to a point set-off from the porous material 310. In the latter example, the interior channel of the cap 340 may define a reservoir 320 therein.

The reservoir 320 is an unoccupied portion of device such as in the cap 340 and is void of other material. In some examples, the reservoir 320 is defined at least partially by the porous material 310 and the cap 340. During use, the bodily fluids that are in the chamber 304 may flow through the porous material 310 to the reservoir 320. The reservoir 320 may store at least some of the bodily fluids therein and/or position the bodily fluids for removal by the conduit 316. In some examples, at least a portion of the porous material 310 may extend continuously between at least a portion of the opening of the interior channel and chamber 304 to wick any bodily fluids from the opening directly to the reservoir 320.

In some examples (not shown), the fluid impermeable barrier 302 may be disposed on or over the cap 340, such as enclosing the cap 340 within the chamber 304.

In some examples, the sheath 332 may include at least a portion of the conduit 316 therein, such as at least partially disposed in the chamber 304. For example, the conduit 316 may extend from the sheath 332 to a region at least proximate to the opening 306. The inlet of the conduit 316 may be positioned adjacent to the annular base 342. The inlet of the conduit 316 may be positioned to be adjacent or proximate to the gravimetrically low point of the chamber 304, such as adjacent to the annular base 342. For example, the inlet may be co-extensive with or offset from the opening 306. In examples, the inlet may be positioned adjacent to the distal end region 322 of the sheath 332 (e.g., substantially opposite the opening 306).

The proximal end region 324 may be disposed near or on the skin around the male urethral opening (e.g., around the penis) and the inlet of the conduit 316 may be positioned in the proximal end region 324. The outlet of the conduit 316 may be directly or indirectly coupled to a vacuum source. Accordingly, bodily fluids may be removed from the proximal end region 324 of the chamber 304 via the conduit 316.

The base 330, the sheath 332, the cap 340, and the conduit 316 may be attached together using any suitable method. For example, at least two of the base 330, the sheath 332, the cap 340, or the conduit 316 may be attached together using at least one of an interference fit, an adhesive, stitching, welding (e.g., ultrasonic welding), tape, any other suitable method, or combinations thereof.

In some examples (not shown), the fluid collection assembly 300 may have a one piece design, with one or more of the sheath 332, the base 330, and the cap 340 being a single, integrally formed piece.

Also as shown, the conduit 316 may be at least partially disposed with the chamber of a fluid collection assembly. The conduit 316 may extend from the distal end region 322 to the proximal end region 324. For example, the conduit 316 may extend through the cap 340 to a point adjacent to the base 330. The conduit 316 is sized and positioned to be coupled to a fluid storage container or the vacuum source. An outlet of the conduit 316 may be operably coupled to the vacuum source, directly or indirectly. The inlet of the conduit 316 may be positioned within the chamber 304 such as at a location expected to be at the gravimetrically low point of the fluid collection assembly during use. By positioning the inlet of the conduit 316 in a location expected to be at the gravimetrically low point of the fluid collection assembly when worn by the user, bodily fluids introduced into the chamber 304 may be removed via the conduit 316 to prevent pooling or stagnation of the bodily fluids within the chamber 304.

In some examples, the vacuum source may be remotely located from the fluid collection assembly 300. In such examples, the conduit 316 may be fluidly connected to the fluid storage container, which may be disposed between the vacuum source and the fluid collection assembly 300.

During operation, a male using the fluid collection assembly 300 may discharge bodily fluids (e.g., urine) into the chamber 304. The bodily fluids may pool or otherwise be collected in the chamber 304. At least some of the bodily fluids may be pulled through the interior of the conduit 316 via the inlet. The bodily fluids may be drawn out of the fluid collection assembly 300 via the vacuum/suction provided by the vacuum source.

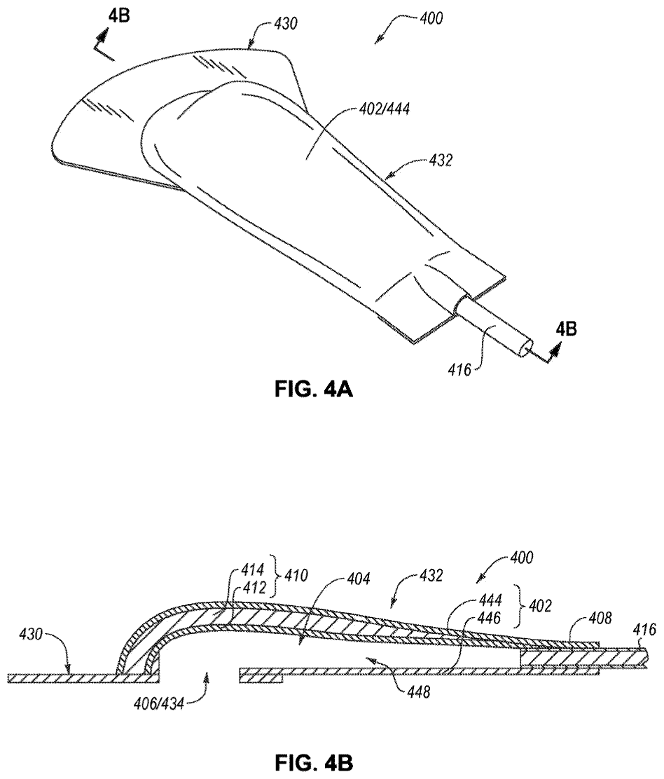

The porous materials disclosed herein may be used with fluid collection assemblies other than the fluid collection assemblies illustrated in FIGS. 1A-3. FIG. 4A is an isometric view of a fluid collection assembly 400, according to an embodiment. FIG. 4B is a cross-sectional schematic of the fluid collection assembly 400 taken along plane 4B-4B. The fluid collection assembly 400 is an example of a male fluid collection assembly though, in some embodiments, the fluid collection assembly 400 may be used to receive bodily fluids from a female urethral opening. The fluid collection assembly 400 includes a sheath 432 and a base 430. The base 430 is configured to be attached (e.g., permanently attached to or configured to be permanently attached) to the sheath 432. The base 430 is also configured to be attached to the region about the urethral opening (e.g., penis) of the individual.

The sheath 432 includes a fluid impermeable barrier 402 that is at least partially formed from a first panel 444 and a second panel 446. The first panel 444 and the second panel 446 may be attached or integrally formed together (e.g., exhibits single piece construction). In an embodiment, as illustrated, the first panel 444 and the second panel 446 are distinct sheets. The fluid impermeable barrier 402 also defines a chamber 404 between the first panel 444 and the second panel 446, an opening 406 at a proximal end region PER of the sheath 432, and a fluid outlet 408 at a distal end region DER of the sheath 432. The sheath 432 also includes at least one porous material 410 disposed in the chamber 404.

The inner surface(s) of the fluid impermeable barrier 402 (e.g., inner surfaces of the first and second panels 444, 446 at least partially defines the chamber 404 within the fluid collection assembly 400. The fluid impermeable barrier 402 temporarily stores the bodily fluids in the chamber 404. The fluid impermeable barrier 402 may be formed from any of the fluid impermeable materials disclosed herein. As such, the fluid impermeable barrier 402 substantially prevents the bodily fluids from passing through the fluid impermeable barrier 402.

In an embodiment, at least one of the first panel 444 or the second panel 446 is formed from an at least partially transparent fluid impermeable material, such as polyethylene, polypropylene, polycarbonate, or polyvinyl chloride. Forming at least one of the first panel 444 or the second panel 446 from an at least partially transparent fluid impermeable material allows a person (e.g., medical practitioner) to examiner the penis. For example, the chamber 404 may include a penis receiving area 448 that is configured to receive the penis of the individual when the penis extends into the chamber 404. The penis receiving area 448 may be defined by at least the porous material 410 and at least a portion of the at least partially transparent material of the first panel 444 and/or the second panel 446. In other words, the porous material 410 is positioned in the chamber 404 such that the porous material 410 is not positioned between the penis and at least a portion of the transparent portion of the first panel 444 and/or second panel 446 when the penis is inserted into the chamber 404 through the opening 406. The porous material 410 is generally not transparent and, thus, the portion of the at least partially transparent material of the first panel 444 and/or the second panel 446 that defines the penis receiving area 448 forms a window which allows the person to view into the penis receiving area 448 and examine the penis.

The opening 406 defined by the fluid impermeable barrier 402 provides an ingress route for bodily fluids to enter the chamber 404 when the penis is a buried penis and allow the penis to enter the chamber 404 (e.g., the penis receiving area 448) when the penis is not buried. The opening 406 may be defined by the fluid impermeable barrier 402 (e.g., an inner edge of the fluid impermeable barrier 402). For example, the opening 406 is formed in and extends through the fluid impermeable barrier 402 thereby enabling bodily fluids to enter the chamber 404 from outside of the fluid collection assembly 400.

The fluid impermeable barrier 402 defines the fluid outlet 408 sized to receive the conduit 416. The conduit 416 may be at least partially disposed in the chamber 404 or otherwise in fluid communication with the chamber 404 through the fluid outlet 408. The fluid outlet 408 may be sized and shaped to form an at least substantially fluid tight seal against the conduit 416 thereby substantially preventing the bodily fluids from escaping the chamber 404. In an embodiment, the fluid outlet 408 may be formed from a portion of the first panel 444 and the second panel 446 that are not attached or integrally formed together. In such an embodiment, the fluid impermeable barrier 402 may not include a cap exhibiting a rigidity that is greater than the portions of the fluid impermeable barrier 402 thereabout which may facilitate manufacturing of the fluid collection assembly 400 may decreasing the number of parts that are used to form the fluid collection assembly 400 and may decrease the time required to manufacture the fluid collection assembly 400. The lack of the cap may make securing the conduit 416 to the fluid outlet 408 using interference fit to be difficult though, it is noted, attaching the conduit 416 to the fluid outlet 408 may still be possible. As such, the conduit 416 may be attached to the fluid outlet 408 (e.g., to the first and second panels 444, 446) using an adhesive, a weld, or otherwise bonding the fluid outlet 408 to the fluid outlet 408. Attaching the conduit 416 to the fluid outlet 408 may prevent leaks and may prevent the conduit 416 from inadvertently becoming detached from the fluid outlet 408. In an example, the conduit 416 may be attached to the fluid outlet 408 in the same manufacturing step that attaches the first and second panels 444, 446 together.

As previously discussed, the sheath 432 includes at least one porous material 410 disclosed in the chamber 404. The porous material 410 may direct the bodily fluids to one or more selected regions of the chamber 404, such as away from the penis and towards the fluid outlet 408. The porous material 410 may be formed from any of the porous materials disclosed herein. In an example, the porous material 410 may be formed from a single layer, two layers (e.g., an outer layer 412 and an inner layer 414), or three or more layers (e.g., an outer layer, an inner layer, and at least one additional layer).

In an embodiment, the porous material 410 may be a sheet. Forming the porous material 410 as a sheet may facilitate the manufacturing of the fluid collection assembly 400. For example, forming the porous material 410 as a sheet allows the first panel 444, the second panel 446, and the porous material 410 to each be sheets. During the manufacturing of the fluid collection assembly 400, the first panel 444, the second panel 446, and the porous material 410 may be stacked and then attached to each other in the same manufacturing step. For instance, the porous material 410 may exhibit a shape that is the same size or, more preferably, slightly smaller than the size of the first panel 444 and the second panel 446. As such, attaching the first panel 444 and the second panel 446 together along the outer edges thereof may also attach the porous material 410 to the first panel 444 and the second panel 446. The porous material 410 may be slightly smaller than the first panel 444 and the second panel 446 such that the first panel 444 and/or the second panel 446 extend around the porous material 410 such that the porous material 410 does not form a passageway through the fluid impermeable barrier 402 through which the bodily fluids may leak. Also, attaching the porous material 410 to the first panel 444 and/or the second panel 446 may prevent the porous material 410 from significantly moving in the chamber 404, such as preventing the porous material 410 from bunching together near the fluid outlet 408. In an example, the porous material 410 may be attached to the first panel 444 or the second panel 446 (e.g., via an adhesive) before or after attaching the first panel 444 to the second panel 446. In an example, the porous material 410 may merely be disposed in the chamber 404 without attaching the porous material 410 to at least one of the first panel 444 or the second panel 446. In an embodiment, the porous material 410 may exhibit shapes other than a sheet, such as a hollow generally cylindrical shape.

Generally, the sheath 432 is substantially flat when the penis is not in the penis receiving area 448 and the sheath 432 is resting on a flat surface. The sheath 432 is substantially flat because the fluid impermeable barrier 402 is formed from the first panel 444 and the second panel 446 instead of a generally tubular fluid impermeable barrier. Further, as previously discussed, the porous material 410 may be a sheet, which also causes the sheath 432 to be substantially flat. The sheath 432 may also be substantially flat because the fluid collection assembly 400 may not include relatively rigid rings or caps that exhibit a rigidity that is greater than the portions of the fluid impermeable barrier 402 thereabout since such rings and caps may inhibit the sheath 432 being substantially flat. It is noted that the sheath 432 is described as being substantially flat because at least one of the porous material 410 may cause a slight bulge to form in the sheath 432 depending on the thickness of the porous material 410, the fluid outlet 408 and/or conduit 416 may cause a bulge thereabout, or the base 430 may pull on portions of the sheath 432 thereabout. It is also noted that the sheath 432 may also be compliant and, as such, the sheath 432 may not be substantially flat during use since, during use, the sheath 432 may rest on a non-flat surface (e.g., may rest on the testicles, the perineum, and/or between the thighs) and the sheath 432 may conform to the surface of these shapes.

The ability of the sheath 432 to be substantially flat when the penis is not in the penis receiving area 448 and the sheath 432 is resting on a flat surface allows the fluid collection assembly 400 to be used with a buried and a non-buried penis. For example, when the fluid collection assembly 400 is being used with a buried penis, the penis does not extend into the penis receiving area 448 which causes the sheath 432 to lie relatively flat across the aperture 434 of the base 430. When the sheath 432 lies relatively flat across the aperture 434, the porous material 410 extends across the opening 406 and the aperature 434 and is in close proximity to the buried penis. As such, the porous material 410 prevents or inhibits pooling of bodily fluids discharged from the buried penis against the skin of the individual since the porous material 410 will receive and remove at least a significant portion of the bodily fluids that would otherwise pool against the skin of the individual. Thus, the skin of the individual remains dry thereby improving comfort of using the fluid collection assembly 400 and preventing skin degradation. However, unlike other conventional fluid collection assemblies that are configured to be used with buried penises, the fluid collection assembly 400 may still be used with a non-buried penis since the non-buried penis can still be received into the penis receiving area 448, even when the penis is fully erect. Additionally, the ability of the sheath 432 to be substantially flat allows the fluid collection assembly 400 to be used more discretely than if the sheath 432 was not substantially flat thereby avoiding possibly embarrassing scenarios.

When the sheath 432 is substantially flat, the porous material 410 occupies substantially all of the chamber 404 and the penis receiving area 448 is collapsed (shown as being non-collapsed in FIG. 4B for illustrative purposes to show the penis receiving area 448). In other words, the sheath 432 may not define a region that is constantly unoccupied by the porous material 410. When the porous material 410 occupies substantially all of the chamber 404, the bodily fluids discharged into the chamber 404 are unlikely to pool for significant periods of time since pooling of the bodily fluids may cause sanitation issues, cause an odor, and/or may cause the skin of the individual to remain in contact with the bodily fluids which may cause discomfort and skin degradation.

As previously discussed, the first panel 444, the second panel 446, and the porous material 410 may be selected to be relatively flexible. The first panel 444, the second panel 446, and the porous material 410 are relatively flexible when the first panel 444, the second panel 446, and the porous material 410, respectively, are unable to maintain their shape when unsupported. The flexibility of the first panel 444, the second panel 446, and the porous material 410 may allow the sheath 432 to be substantially flat, as discussed above. The flexibility of the first panel 444, the second panel 446, and the porous material 410 may also allow the sheath 432 to conform to the shape of the penis even when the size and shape of the penis changes (e.g., becomes erect) and to minimize any unoccupied spaces in the chamber 404 in which bodily fluids may pool.

As previously discussed, the fluid collection assembly 400 includes a base 430 that is configured to be attached to the sheath 432. For example, the base 430 is configured to be permanently attached to the sheath 432. The base 430 is configured to be permanently attached to the sheath 432 when, for example, when the fluid collection assembly 400 is provided with the base 430 permanently attached to the sheath 432 or the base 430 is provided without being permanently attached to the sheath 432 but is configured to be permanently attached to the sheath 432 at some point in the future. Permanently attached means that the sheath 432 cannot be detached from the base 430 without damaging at least one of the sheath 432 or the base 430, using a blade to separate the sheath 432 from the base 430, and/or using chemicals to dissolve the adhesive that attaches the sheath 432 from the base 430. The base 430 may be permanently attached to the sheath 432 using an adhesive, sewing, heat sealing, radio frequency welding, or ultrasonic welding. In an embodiment, the base 430 is configured to be reversibly attached to the sheath 432. In an embodiment, the base 430 is integrally formed with the sheath 432.

The base 430 includes an aperture 434. The base 430 is permanently attached to the sheath 432 such that the aperture 434 is aligned with the opening 406. The base 430 is sized, shaped, and made of a material to be coupled to the skin that surrounds the penis (e.g., mons pubis, thighs, testicles, and/or perineum) and have the penis disposed therethrough. For example, the base 430 may define an aperture 434 configured to have the penis positioned therethrough. In an example, the base 430 may exhibit the general shape or contours of the skin surface that the base 430 is configured to be coupled with. The base 430 may be flexible, thereby allowing the base 430 to conform to any shape of the skin surface and mitigate the base 430 pulling the on skin surface. The base 430 may extend laterally past the sheath 432 thereby increasing the surface area of the skin of the individual to which the fluid collection assembly 400 may be attached compared to a substantially similar fluid collection assembly 400 that did not include a base.

As previously discussed, the fluid collection assembly 400 includes the conduit 416. The inlet of the conduit 416 may be located near the distal end region DER of the sheath 432 which is expected to be the gravimetrically low point of the chamber 404 when worn by an individual.

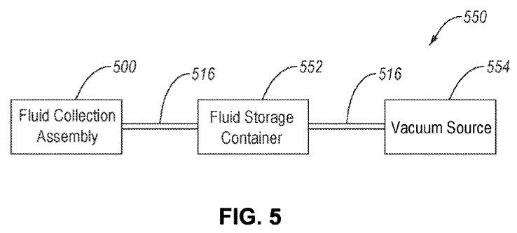

FIG. 5 is a block diagram of a fluid collection system 550 for fluid collection, according to an embodiment. The fluid collection system 550 includes a fluid collection assembly 500, a fluid storage container 552, and a vacuum source 554. The fluid collection assembly 500 may be the same or substantially similar to any of the fluid collection assemblies disclosed herein. The fluid collection assembly 500, the fluid storage container 552, and the vacuum source 554 may be fluidly coupled to each other via one or more conduits 516. For example, fluid collection assembly 500 may be operably coupled to one or more of the fluid storage container 552 or the vacuum source 554 via the conduit 516. The bodily fluids collected in the fluid collection assembly 500 may be removed from the fluid collection assembly 500 via the conduit 516 which protrudes into the fluid collection assembly 500. For example, an inlet of the conduit 516 may extend into the fluid collection assembly 500, such as to a reservoir therein. The outlet of the conduit 516 may extend into the fluid collection assembly 500 or the vacuum source 554. Suction force may be introduced into the chamber of the fluid collection assembly 500 via the inlet of the conduit 516 responsive to suction (e.g., vacuum) force applied at the outlet of the conduit 516.

The suction force may be applied to the outlet of the conduit 516 by the vacuum source 554 either directly or indirectly. The suction force may be applied indirectly via the fluid storage container 552. For example, the outlet of the conduit 516 may be disposed within the fluid storage container 552 and an additional conduit 516 may extend from the fluid storage container 552 to the vacuum source 554. Accordingly, the vacuum source 554 may apply suction to the fluid collection assembly 500 via the fluid storage container 552. The suction force may be applied directly via the vacuum source 554. For example, the outlet of the conduit 516 may be disposed within the vacuum source 554. An additional conduit 516 may extend from the vacuum source 554 to a point outside of the fluid collection assembly 500, such as to the fluid storage container 552. In such examples, the vacuum source 554 may be disposed between the fluid collection assembly 500 and the fluid storage container 552.

The fluid storage container 552 is sized and shaped to retain bodily fluids therein. The fluid storage container 552 may include a bag (e.g., drainage bag), a bottle or cup (e.g., collection jar), or any other enclosed container for storing bodily fluids such as urine. In some examples, the conduit 516 may extend from the fluid collection assembly 500 and attach to the fluid storage container 552 at a first point therein. An additional conduit 516 may attach to the fluid storage container 552 at a second point thereon and may extend and attach to the vacuum source 554. Accordingly, a vacuum (e.g., suction) may be drawn through fluid collection assembly 500 via the fluid storage container 552. Bodily fluids, such as urine, may be drained from the fluid collection assembly 500 using the vacuum source 554.

The vacuum source 554 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 554 may provide a vacuum or suction to remove bodily fluids from the fluid collection assembly 500. In some examples, the vacuum source 554 may be powered by one or more of a power cord (e.g., connected to a power socket), one or more batteries, or even manual power (e.g., a hand operated vacuum pump). In some examples, the vacuum source 554 may be sized and shaped to fit outside of, on, or within the fluid collection assembly 500. For example, the vacuum source 554 may include one or more miniaturized pumps or one or more micro pumps. The vacuum sources 554 disclosed herein may include one or more of a switch, a button, a plug, a remote, or any other device suitable to activate the vacuum source 554.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable barrier at least defining:
a chamber;
at least one opening; and
a fluid outlet;
at least one porous material disposed in the chamber, the at least one porous material including an inner layer and an outer layer disposed on the inner layer, the inner layer including a polyethylene foam and the outer layer including a polyurethane foam.

2. The fluid collection assembly of claim 1, wherein the polyurethane foam is hydrophilic.

3. The fluid collection assembly of claim 1, wherein the polyurethane foam exhibits an average of 6 pores/cm² to 14 pores/cm².

4. The fluid collection assembly of claim 1, wherein the polyurethane foam exhibits an average density of about 100 kg/m³ to about 170 kg/m³.

5. (canceled)

6. (canceled)

7. The fluid collection assembly of claim 1, wherein the polyethylene foam is hydrophobic.

8. The fluid collection assembly of claim 1, wherein the polyethylene foam exhibits an average of about 12 pores/cm² to about 28 pores/cm².

9. The fluid collection assembly of claim 1, wherein the polyethylene foam exhibits a pores/cm² that is greater than the polyurethane foam.

10. The fluid collection assembly of claim 1, wherein the polyethylene foam exhibits a density of about 70 kg/m³ to about 150 kg/m³.

11. The fluid collection assembly of claim 1, wherein the polyethylene foam exhibits a number of pores/cm² that is greater than a number of pores/cm² of the polyurethane foam.

12. The fluid collection assembly of claim 1, wherein the at least one porous material includes one or more additional layers disposed on the polyurethane foam.

13. The fluid collection assembly of claim 1, wherein the polyurethane foam extends across the at least one opening.

14. The fluid collection assembly of claim 1, wherein the fluid collection assembly is configured to receive bodily fluids from a female urethral opening.

15. The fluid collection assembly of claim 1, wherein the fluid collection assembly is configured to received one or more bodily fluids from a male urethral opening.

16. The fluid collection assembly of claim 1, wherein the fluid collection assembly may be used for a period of time greater than 24 hours.

17. A fluid collection assembly, comprising:
a fluid impermeable barrier at least defining:
a chamber;
at least one opening; and
a fluid outlet;
at least one porous material disposed in the chamber, the at least one porous material including an inner layer and an outer layer disposed on the inner layer, the inner layer including a hydrophobic polyethylene foam and the outer layer including a hydrophilic polyurethane foam.

18. (canceled)

19. (canceled)

20. A fluid collection system, comprising:
a fluid collection assembly including:
a fluid impermeable barrier at least defining:
a chamber;
at least one opening; and
a fluid outlet;
at least one porous material disposed in the chamber, the at least one porous material including an inner layer and an outer layer disposed on the inner layer, the inner layer including a polyethylene foam and the outer layer including a polyurethane foam a fluid storage container; and
a vacuum source;
wherein the chamber of the fluid collection assembly, the fluid storage container, and the vacuum source are in fluid communication with each other such that a suction provided to the chamber from the vacuum source removes one or more bodily fluids from the chamber and deposits the one or more bodily fluids in the fluid storage container.

21. The fluid collection assembly of claim 1, wherein the outer layer exhibits a thickness of about 0.4 mm or less.

22. The fluid collection assembly of claim 1, wherein the polyurethan foam exhibits a density that is greater than the polyethylene foam.

23. The fluid collection assembly of claim 17, wherein:
the polyethylene foam exhibits a number of pores/cm² that is greater than a number of pores/cm² of the polyurethane foam; and
the polyurethan foam exhibits a density that is greater than the polyethylene foam.

24. The fluid collection assembly of claim 17, wherein:
the polyurethane foam exhibits an average of about 6 pores/cm² to about 14 pores/cm² and an average density of about 100 kg/m³ to about 170 kg/m³; and
the polyethylene foam exhibits an average of about 12 pores/cm² to about 28 pores/cm² and a density of about 70 kg/m³ to about 150 kg/m³.
